(19) United States
(12) Patent Application Publication
(10) Pub. No.: US 2004/0092036 A1
(43) Pub. Date: May 13, 2004

(54) Device for analysing analyte compounds and use hereof

(75) Inventors: Fei Chen, Horsholm (DK); Ann Merete Moller, Bagsvaerd (DK)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(73) Assignee: LATTEC I/S

(21) Appl. No.: 10/659,367

(22) Filed: Sep. 11, 2003

Related U.S. Application Data
(60) Provisional application No. 60/409,567, filed on Sep. 11, 2002.

(51) Int. Cl. G01N 33/558

(52) U.S. Cl. 436/514

(57) ABSTRACT

An analytical device consisting of porous material(s) that permit(s) liquid to migrate therein, the device comprising In the migration direction. The device comprises a first zone onto which a sample suspected of containing an analyte to be assayed can be applied, a second zone incorporating a non-immobilised molecule capable of specifically binding to the analyte, said molecule is provided with a detectable label, a third zone capable of retarding the rate of migration of the sample and the non-immobilised molecule, and a fourth zone incorporating in at least part of the zone an immobilised state the same type of analyte as the one to be assayed or an analogue thereof being capable of specifically binding to the non-immobilised molecule.

### FIELD OF INVENTION

The present invention relates in general to the field of analysing analytes in biological samples. In particular, a novel competitive immunoassay device for assaying steroid compounds in such samples, including milk samples, with the objective of improving reproductive performance of dairy herds is provided.

### TECHNICAL BACKGROUND AND PRIOR ART

One of the major factors affecting the profitability of dairy enterprises is the calving interval. A calving interval of 365 days is ideal. Poor reproductive performance results in longer calving intervals, which evidently have a negative impact on profitability. The variability in the economic impact of reproductive performance can be inferred from the marked differences between selected countries in respect of conception rates relative to first insemination. In New Zealand, for example, where calving according to season is important, an average conception rate of 85% has been reported. In Great Britain, where milk production from grazed grass is less financially important, the average conception rate is currently down to around 40%.

Over the last decade there has been a dramatic decline in the average reproductive performance. The interval between calvings has increased by 20 days and the number of cows conceiving after first insemination has dropped by 15%. Although there has been substantial changes in dairy management over this period, such as increases in herd size and increasing automation, it is implausible that the decline in reproductive performance can be ascribed solely to a universal worsening in the reproductive management ability of dairy farmers. Over a similar time span, it has been shown that genetic selection for milk yield has resulted in decreased reproductive performance at a genetic level. There is a strong association between milk production potential of the cows, body energy mobilisation and decreased reproductive performance. Thus the current poor reproductive performance is not only costly but also on the increase.

The key factors for achieving an optimal calving interval include the length of post-calving anoestrus, precise oestrus detection and rapid follow-up on cows not conceiving at the first insemination. Biologically speaking, an extended post-calving anoestrus period indicates that the cow is experiencing metabolic stress in early lactation, typically mobilising excessive amounts of body energy reserves. Other stress factors, such as disease and social competition, can also affect the length of the post-calving anoestrus. However, in the absence of physiological measures, extended anoestrus periods may merely reflect a failure to detect first oestrus. Once the cow is cycling, the interval from first oestrus to conception is largely determined by reproductive management skill.

Precise detection of oestrus, and thus correct timing of insemination, has a substantial effect on the subsequent conception rate. For the cows that do not conceive, the earliest possible recognition hereof reduces the delay in getting those particular cows bred again.

Currently, determining the point in time during oestrus, where insemination is most likely to result in conception is generally based on visual inspection of heat manifestations that may give a rate and an accuracy of oestrus detection in excess of 90%. However, this is possible only when skilled personnel is used and only if a substantial amount of time is devoted to this activity. Accordingly, reliable objective physiological indicators of reproductive status would be of considerable value. Given examples are: milk temperature profiles have been suggested, physical activity tags or combined observations of temperatures and physical activity are such indicators. However, none of these indicators are entirely reliable indicators of oestrus.

A further significant prerequisite for securing optimal reproductive performance of a dairy herd is the ability to determine that conception has been achieved as early as possible after insemination. An unsuccessful conception must be followed up by repeated insemination.

Detection of pregnancy in cows is currently made by rectal palpation, but this method is only reliable when applied 5 to 6 weeks after insemination. In most cases, cows that have failed to conceive are defined as those showing oestrus signs about 21 or 42 days after insemination. However, this approach relies upon good oestrus detection skills. It has been reported that re-insemination of already pregnant cows may occur in up to 20% of second inseminations. This is an unnecessary cost which also involves a risk of inducing abortion of the original conceptus. An objective and reliable early measure of pregnancy would thus be a useful reproductive management aid.

A number of studies have attempted to measure molecules that are pregnancy specific, such as oestrone sulphate (only after day 45), pregnancy-specific proteins and pregnancy-associated glycoprotein. However, interpretation of these measures has not yet proved to be entirely straightforward, partly because there can be many different aetiologies of early embryo loss. A more promising alternative is β-mode ultrasonography, whereby foetal heartbeat has been detected as early as 20 days after conception.

Several attempts have been made to identify compounds indicating the state in the reproductive cycle. The detection of such compounds in milk may be reliable indicators of reproductive performance, including oestrus detection and detection of conception. One such compound, whose usefulness for these purposes has been investigated, is progesterone. Progesterone is a steroid hormone that is mainly produced by the corpus luteum and to some extent the placenta of the pregnant cow. It is found in ng/ml concentrations in plasma. There is a close correlation between plasma progesterone and milk progesterone (r=0.90; Abeyawandene et al., 1984). Progesterone, being a steroid hormone, is fat-soluble and can, consequently, be found in different concentrations in defatted milk and milk fat. Defatted milk concentrations of progesterone are approximately 50% of plasma concentrations. Milk fat concentrations of progesterone are 5 to 10 times higher than plasma concentrations.

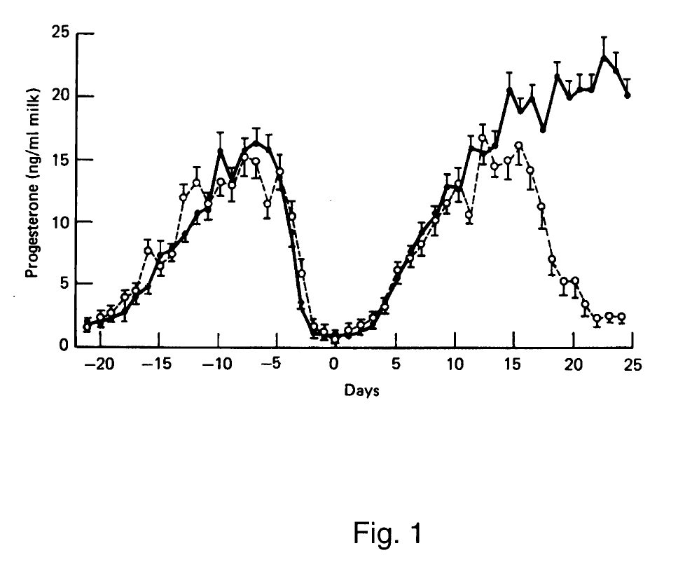

Whole milk progesterone concentrations are between those of defatted milk and milk fat and reflect the relative proportions of these two milk fractions. Consequently, milk progesterone concentrations have been found to vary with milk fat content (Waldmann, 1993). A typical pattern of whole milk progesterone throughout one oestrus cycle and the onset of a subsequent pregnancy is shown in FIG. 1. The important feature is the large difference between progesterone concentrations in the luteal phase (days −16 to −4) and progesterone in the period around oestrus (days −3 to +3). Detecting this difference may form the basis not only for oestrus detection but also pregnancy detection and detection of length of the postpartum anoestrus.

Using progesterone, pregnancy detection would rely upon differentiating between the normal progesterone pattern of a non-pregnant cycling cow and the pattern of the pregnant cow. As can be seen in FIG. 1, this could, in theory, occur from approximately 15 days after a correctly timed insemination. The progesterone level of the cycling cow starts to decline as she comes into oestrus, whilst those of the pregnant cow remains high. The necessary precision to distinguish pregnant from non-pregnant cows, in an individual progesterone determination decreases with increasing days from insemination until the progesterone pattern of the following cow cycle starts raising again at about 24 days after oestrus. However, the main purpose of progesterone measurements after insemination is to detect non-pregnant cows which require re-insemination. Most literature reports indicate that reliable differentiation of pregnant from non-pregnant cows occurs around day 20 after conception.

Oestrus periods can be reliably identified from progesterone patterns, where samples are taken every second or third day (Royal et al., 2000). However, progesterone levels are low throughout the period from 2-3 days before until 2-3 days after oestrus, and successful insemination depends upon precise identification of oestrus itself. Salisbury and VanDemark (1961) showed that timing of insemination relative to oestrus had a dramatic effect on pregnancy rates. The optimum time for insemination is 4-8 hours after ovulation. Furthermore, individual cows show considerable variation in both the time interval between the drop in progesterone levels and the onset of oestrus as well as in the duration of oestrus itself.

Generally, conventional assays for detecting analytes in biological samples include competitive immunoassays and immunoassays based on the “sandwich” technique.

Generally, competitive immunoassays are based on designs where an antibody against the analyte is immobilised on a solid support and a labelled analyte is added to the sample. If a non-labelled analyte is present in the sample, this native analyte and the labelled analyte will compete for binding sites on the immobilised antibody. However, this approach is associated with several problems: it is technically difficult to label relatively small analytes. The presence of a relatively large labelling substance may result in steric hindrance of the steroid-antibody binding for instance, in turn resulting in a less than optimal sensitivity of the assay.

The above problem arising from poor coupling performance in such conventional competitive immunoassays may be overcome by using a “sandwich” type immunoassay. In such an assay format, an antibody against the analyte to be analysed is immobilised on a solid support and a labelled antibody against the analyte is applied to the sample. The analyte is bound between the two antibodies. One problem associated with “sandwich” immunoassays is that this assay format is most suited for the detection of analytes that have at least two antigenic sites. Additionally, this type of immunoassays requires a relatively high amount of antibodies and therefore, becomes more expensive.

In prior art such as U.S. Pat. No. 6,001,658, EP 0 895 084 and EP 1 061 369, many examples of constructing immunoassays or devices for detecting an analyte in a sample has been disclosed. The disadvantage of these disclosed assays is that they do not involve a zone in the device or a compound specifically designed to delay or retard the migration of the sample.

EP 0 810 436 discloses a competitive immunoassay device for detecting an analyte in a liquid sample. The assay may preferably be used as a preagnancy test, based on the addition of the sample to a nitrocellulose membrane releasing a labelled mobile specific binding reagent against the analyte to be assayed. The liquid sample is assayed adding a viscosity modifier capable of slowing down the migration followed by applying mixture to the nitrocellulose membrane. The disadvantage of this assay device is the additional step of adding viscosity modifier to the sample before assaying making the device complicated to use for the unskilled user.

As described above, measurements of progesterone in milk samples are potentially useful means of optimising reproductive performance, provided that analytical methods which permits sensitive, precise and reproducible detection of very small quantities of the steroid that is in the range of 0-50 ng/ml are available. Thus, it is a requirement for such an assay that it allows very small day-to-day changes in the level of progesterone to be detected, particularly around oestrus and around the point in time where pregnancy and non-pregnancy levels begin to deviate.

Accordingly, a suitable assay for monitoring progesterone levels on a daily basis or during selected time intervals must be cheap, highly sensitive, precise, reproducible, easy to perform, require a minimum of handling steps and preferably be compatible with automated and semi-automated systems for optimising performances of milk producing herds, such as the system disclosed in co-pending European patent application No. 01610022.4. Such an analytical method and the means for performing the method are provided herein.

### SUMMARY OF THE INVENTION

Accordingly, the present invention provides a device and a method for quantitative determination of an analyte in a sample such as milk. The present invention makes it possible to monitor an analyte of interest on a daily basis or during selected time intervals in a way which is cheap, highly sensitive, reproducible, precise, requires a minimum of handling steps and which is easy to perform.

According to the present invention, the device permits the sample after being applied to a first zone to migrate through a second zone, thereby releasing a specific binding molecule from said second zone. The specific binding molecule is able to specifically bind the analyte to be assayed in the sample. Upon release of the specific binding molecule, the sample now containing the specific binding molecule migrates through a third zone in which the migration of the sample and the specific binding molecule is delayed in order to extend the time for analyte and specific binding molecule to combine. After passing the third zone the sample and the specific binding molecule comes to a fourth zone. When passing the fourth zone, any non-bound specific binding molecule is bound to molecules similar to the analyte or analogue thereof which have been immobilised in the detection site located in the fourth zone. Excess of analyte specific binding molecule is thereby bound and the amount of this excess of analyte specific binding molecule is rendered measurable.

Using either an internal and/or external reference or calibration enables the quantitative determination of the analyte to be assayed and which is present in the liquid sample.

### DETAILED DISCLOSURE OF THE INVENTION

Thus, in the broadest aspect of the present invention, an analytical device consisting of porous material(s) that permit(s) liquid to migrate therein is provided, the device comprising in the migration direction:

(i) a first zone onto which a sample suspected of containing an analyte to be assayed can be applied,

(ii) a second zone incorporating a non-immobilised molecule capable of specifically binding to the analyte, said non-immobilised molecule is provided with a detectable label,

(iii) a third zone capable of retarding the rate of migration of the sample and the non-immobilised molecule, and

(iv) a fourth zone incorporating in at least part of the zone an immobilised state the same type of analyte as the one to be assayed or an analogue thereof being capable of specifically binding to the non-immobilised molecule.

Furthermore in the broadest aspect of the present invention, a method for assaying an analyte in a sample is provided, the method comprising the steps of:

(i) applying the sample suspected of containing an analyte to a first zone,

(ii) permitting the sample to migrate through a second zone incorporating a non-immobilised molecule capable of specifically binding to the analyte, said non-immobilised molecule is provided with a detectable label,

(iii) permitting the sample to migrate through a third zone incorporating at least one substance, the presence of which retard the rate of migration of the sample and the non-immobilised molecule, and

(iv) permitting the sample to migrate through a fourth zone incorporating in at least part of the zone an immobilised state the same type of analyte as the one to be assayed or an analogue thereof being capable of specifically binding to the non-immobilised molecule.

Under assaying conditions, a known amount of the sample is added to the first zone and permitted to migrate from the first zone through the second zone towards and through the third zone. The migration proceeds towards and through the fourth zone, after which any unbound material is adsorbed in an fifth zone located downstream of the fourth zone and providing a force to the sample to direct the migration through the first zone, the second zone, the third zone and the fourth zone. The quantitative determination of the analyte to be assayed can be determined by a relation between the signal intensity from the detection site located in the fourth zone given by the label specifically bound to the fourth zone and by an external and/or an internal reference. In accordance with the present invention, this provides a suitable assay for monitoring small levels of analyte for instance on a daily basis or during selected time intervals in a cheap, highly sensitive, reproducible, precise and easy manner.

In accordance with the present invention, the device comprises a porous material wherein a sample can migrate. In this porous material the specific binding molecule, the analyte and other non-analyte compounds present in the applied sample are migrating together. In the present context, the term “migrate” relates to the movement of the sample through at least one of the zones, i.e. the first zone, the second zone, the third zone, the fourth zone or the fifth zone. This movement of the sample can be provided by actions such as floating action, capillary action or gravity action. The migration proceeds when the porous material is made wet for instance by the liquid sample.

In the present context, the terms “specifically binding” and “specific binding” relates to the binding between a pair of molecules (each being a member of a specific binding pair) which are naturally derived or synthetically produced. One of the molecules of the pair has an area on its surface or a cavity to which the other molecule of the pair specifically binds, and is therefore defined as complementary with a particular spatial and polar organisation of the other molecule, so that the pair has the property of binding specifically to each other. Examples of types of specific binding pairs are antigen-antibody, biotin-avidin, hormone-hormone receptor, receptor-ligand, enzyme-substrate, IgG-protein A.

Preferably, the device of the present invention is designed to perform quantitative measurements. In one useful embodiment the device may be in the form of test strips (also known as dry sticks).

The porous material

The materials selected to be used in the first zone, the second zone, the third zone, the fourth zone and the fifth zone is selected from a porous material. In present context the term “porous material” relates to a material which adsorbs the sample and thereby permits it to migrate. The porous material selected may comprises a pore-size and has a capacity that makes it possible to provide a high flow-rate which reduces the assay time. Preferably, the porous material is selected for providing substantially no retention of triglyceride rich samples. In an embodiment of the present invention the retention of triglycerides is 0%, such as at the most 1%, e.g. at the most 2.5%, such as at the most 5%, e.g. at the most 10%, such as at the most 15%, in the, e.g. at the most 25%, such as at the most 50%, e.g. at the most 75%, such as at the most 100%.

The porous material is preferably selected from the group consisting of a nitrocellulose membrane, cellulose, a polymer such as nylon, polyvinylidene fluoride or latex, glass fibre, woven fibres, non-woven fibres and a chromatographic gel membranes.

In an embodiment of the present invention, the porous material is selected from a group of materials comprising a pore size preferably in the range of 10-30.000 μm, such as in the range of 10-20.000 μm, for instance in the range of 10-10.000 μm, for instance in the range of 10-1000 μm, such as in the range of 10-500 μm, such as in the range of 10-100 μm, for instance in the range of 10-75 μm, such as in the range of 10-50 μm, for instance in the range of 50-200 μm, such as the range of 50-100 μm, for instance in the range of 100-500 μm, such as the range of 50-300 μm, for instance in the range of 75-300 μm, such as the range of 75-200 μm, for instance in the range of 75-150 μm, such as the range of 75-120 μm.

In yet an embodiment of the present invention, the porous material is selected from a group of materials comprising a suitable pore size such as at most 500 μm, for instance at most 200 μm, such as at most 150 μm, for instance at most 100 μm, such as at most 75 μm.

In another embodiment of the present invention, the porous material is characterised by having a high capacity of binding proteins such as in the range of 1-400 μg/cm², for instance the range of 1-250 μg/cm², such as the range of 1-200 μg/cm², for instance the range of 1-140 μg/cm², such as the range of 1-120 μg/cm², for instance the range of 1-100 μg/cm², such as the range of 1-80 μg/cm², for instance the range of 1-60 μg/cm², such as the range of 1-40 μg/cm², for instance the range of 50-200 μg/cm², such as the range of 50-100 μg/cm², for instance the range of 50-150 μg/cm², such as the range of 50-120 μg/cm², for instance the range of 75-120 μg/cm², such as the range of 75-110 μg/cm².

In a further embodiment of the present invention, the porous material is characterised by having a high capacity of binding proteins such as at most 400 μg/cm², for instance at most 250 μg/cm², such as at most 200 μg/cm², for instance at most 140 μg/cm², such as at most 120 μg/cm², for instance at most 100 μg/cm², such as at most 80 μg/cm², for instance at most 60 μg/cm², such as at most 40 μg/cm².

Accordingly, in an embodiment of the present invention, the porous material is characterised by permitting the sample to migrate with a high capillary flow rate, such as in the range of 50-500 sec/4 cm, for instance the range of 50-250 sec/4 cm, such as the range of 50-200 sec/4 cm, such as the range of 50-100 sec/4 cm, for instance the range of 50-75 sec/4 cm, such as the range of 100-250 sec/4 cm, for instance the range of 150-250 sec/4 cm, such as the range of 200-250 sec/4 cm and for instance the range of 250-500 sec/4 cm, such as the range of 75-150 sec/4 cm and for instance the range of 80-130 sec/4 cm, such as the range of 80-110.

In another embodiment of the present invention, the porous material is characterised by permitting the sample to migrate with a high capillary flow rate, such as at most 300 sec/4 cm, for instance at most 200 sec/4 cm, such as at most 100 sec/4 cm, such as at most 75 sec/4 cm.

Preferably, the porous materials used in the first zone, the second zone, the third zone, the fourth zone and the fifth zone are the same in at least 5 of the zones, such as at least 4 of the zones, for instance 3 of the zones, such as at least 2 of the zones.

In accordance with the above pore size, capacity and flow-rate, it is desirable to provide a device for detecting an analyte in a fast assay. In an embodiment of the present invention the assay time is less than 15 minutes, such as less than 10 minutes, e.g. less than 8 minutes, such as less than 7 minutes, e.g. less than 6 minutes, such as less than 5 minutes, e.g. less than 4 minutes, such as less than 3 minutes, e.g. less than 2 minutes, such as less than 1 minute, e.g. less than 30 seconds.

The First Zone

Preferably, the device according to the present invention is provided with a first zone. In the present context, the term “first zone” relates to a site in the device where the liquid sample is applied to the device and which provides a fast adsorption of the liquid sample and a fast and consistent release of a sample to the second zone. Accordingly, the material used in the first zone is selected from the group consisting of a nitrocellulose membrane, cellulose, a polymer such as nylon, polyvinylidene fluoride or latex, glass fibre, woven fibres, non-woven fibres and a chromatographic gel membrane. Preferably, the material used in the first zone is a woven or a non-woven glass fibre.

In a preferred embodiment of the present invention, the porous material to be used in the first zone is a cellulose material such as Ahlstrom 8975 from Ahlstrom.

In one embodiment of the present invention, the first zone uses porous materials different from the materials used in at least one of the other zones. This diversity gives the opportunity to provide the first zone with different characteristics than the other zones, for instance different properties in the binding, bed volume, different migration properties of the specific binding conjugate.

In yet an embodiment, the material used can be the same as the material used in at least one of the second zone and/or in the third or fourth zone.

Preferably, the first zone is located upstream from the fourth zone and substantially upstream from the second zone, whereupon the sample migrates through the second zone to release the non-immobilised specific binding molecule.

In an embodiment of the present invention, the first zone and the second zone can be partially overlapping. Preferably, the two zones provide about 1% overlap between the areas of the two zones, about 2% overlap between the areas of the two zones, about 3% overlap between the areas of the two zones, about 3% overlap between the areas of the two zones, about 4% overlap between the areas of the two zones, about 5% overlap between the areas of the two zones, about 10% overlap between the areas of the two zones, about 20% overlap between the areas of the two zones is provided, about 30% overlap between the areas of the two zones is provided, about 40% overlap between the areas of the two zones is provided, about 50% overlap between the areas of the two zones is provided, about 60% overlap between the areas of the two zones is provided, about 70% overlap between the areas of the two zones is provided, about 80% overlap between the areas of the two zones is provided, about 90% overlap between the areas of the two zones is provided or about 100% overlap between the areas of the two zones is provided.

In yet an embodiment, the material used in the first zone provides an appropriate bed volume relative to the sample volume. In the present context, the term “appropriate bed volume relative to sample volume” relates to a bed volume large enough to receive the entire sample applied without any of the sample being lost. In an embodiment of the present invention, the volume of the sample to be applied to and adsorbed by the first zone is preferably at most 1000 μl, such as at most 500 μl, for instance at most 200 μl, such as at most 175 μl, for instance at most 150 μl, such as at most 125 μl, for instance at most 100 μl, such as at most 75 μl, for instance at most 50 μl, such as at most 25 μl, for instance at most 10 μl.

In a further embodiment of the present invention, the volume of the sample to be applied to and adsorbed by the first zone is preferably in the range of 1-1000 μl, such as in the range of 1-500 μl, for instance in the range of 1-200 μl, such as in the range of 1-175 μl, for instance in the range of 1-150 μl, such as in the range of 1-125 μl, for instance in the range of 1-100 μl, such as in the range of 1-75 μl, for instance in the range of 1-50 μl, such as in the range of 1-25 μl, for instance in the range of 1-10 μl, such as in the range of 10-200 μl, for instance in the range of 25-200 μl, such as in the range of 50-200 μl, for instance in the range of 100-200 μl.

The Second Zone

The device described in the present application comprises a second zone. In the present context, “second zone” relates to either an integrated zone in the first zone or a separate zone connected to the first zone. The second zone comprises the non-immobilised specific binding molecule. After the application of the sample, the sample migrates through the said second zone before migrating through the third zone and the fourth zone. The material used in the second zone provides a fast, consistent and quantitative release of both the non-immobilised specific binding molecule and provides low or substantially no retention of the triglyceride or proteins in the sample

In one embodiment of the present invention, the second zone is separated from the first zone and the third zone.

In another embodiment of the present invention the porous material used can be the same as the porous material used in the first zone.

In a preferred embodiment of the present invention, the porous material to be used in the second zone is a cellulose material such as Ahlstrom 8975 from Ahlstrom.

The second zone comprises non-immobilised specific binding molecule. In the present context, the term “specific binding molecule” used interchangeable with the terms “non-immobilised molecule” and “non-immobilised specific binding molecule” refer to a molecule which is freely movable in the moist state. Preferably, when dry, the specific binding molecule is substantially non-movable and when moistened the specific binding molecule is released and starts migrating. In an embodiment of the present invention the specific binding molecule is a protein, an antibody, a receptor, an enzyme, a peptide, an amino acid, a hormone, a vitamin, a drug or a combination thereof which when attached to a label provides the non-immobilised specific binding molecule. Preferably, the antibody used in the specific binding conjugate is a monoclonal antibody specific for binding the analyte to be assayed or an analogue thereof.

In the present context the term “substantially non-movable” relates to the migration of the non-immobilised specific binding molecule within the second zone when in the dry state. The migration of the non-immobilised specific binding molecule within the second zone when in the dry state is less than 100% of the specific binding molecules, such as less than 50%, e.g. less than 25%, such as less than 15%, e.g. less than 10%, such as less than 5%, e.g. less than 2%, such as less than 1%.

The migration of the non-immobilised specific binding molecule out of the second zone when in the dry state is less than 100% of the specific binding molecules, such as less than 50%, e.g. less than 25%, such as less than 15%, e.g. less than 10%, such as less than 5%, e.g. less than 2%, such as less than 1%. Preferably 0% of the non-immobilised specific binding molecule migrates in the dry state.

In the present context, the term “label” refers to any substance which directly or indirectly is attached to the specific binding molecule and is capable of producing a signal that is detectable by visual or instrumental means. The instrumental means may be e.g. magnotometer, spectrophotometer, ELISA-reader. Various suitable labels for use in the present invention are selected from the group consisting of chromogens, catalysts, fluorescent compounds, chemiluminescent compounds, radioactive labels, metals, magnetic particles, dye particles, enzymes or substrates, or organic polymer latex particles; liposomes or other vesicles containing signal producing substances and the like.

In a preferred embodiment of the present invention, the label can be selected from the group of metals consisting of gold and silver but also other elements like carbon, fluorescent latex beads and dyed latex beads can be used, where no additional manipulation or at least a minimum of manipulation of the label is required to produce a detectable signal.

In another preferred signal producing system, the label can be a fluorescent compound where no enzymatic manipulation of the label is required to produce a detectable signal.

In another preferred embodiment of the present invention, a visually detectable, coloured particle can be used as the label component of the indicator reagent, thereby providing for a direct colour readout of the presence or concentration of the analyte in the sample without the need for addition of other signal producing reagents.

In a preferred embodiment of the present invention the ratio between non-immobilised molecule and the detectable label is in the range of 1:99 respectively, such as in the range of 25:75 respectively, e.g. in the range of 50:50, such as in the range of 75:25 respectively and e.g. in the range of 99:1 respectively.

The Third Zone

In a preferred embodiment of the present invention, the device according to the present invention is provided with a third zone. In the present context, the term “third zone” refers to a site preferably located between the second zone and the fourth zone. In a preferred embodiment of the present invention the third zone incorporating at least one substance the presence of which retard the rate of migration of the sample and the specific binding molecule. Thereby the migration rate of the sample and the released specific binding molecule has been reduced whereby the sample and the released specific binding molecule have more time to mix and react.

In the present context the term “at least one substance the presence of which retard the rate of migration of the sample and the specific binding molecule” relates to the parameters involved in increasing the time of reaction/incubation which can be achieved by the incorporating viscosity modifiers (e.g. sugars, proteins (e.g. BSA) and modified celluloses) in the third zone to slow down the reagent migration.

In a preferred embodiment of the present invention the retarded rate of migration in the third zone is provided in order to increasing the time of reaction/incubation between the analyte and the specific binding molecule.

In another embodiment of the present invention the retention feature provided in the third zone is provided by the selection of porous material having features which by it self is capable of retarding the sample and the specific binding molecule such as changing the lenght of the porous material used in the third zone or changing the porosity of the porous material. Alternatively said porous material could be used in combination with the at least one substance the presence of which also retard the rate of migration of the sample and the specific binding molecule.

In an embodiment of the present invention, the third zone and the second zone can be partially overlapping. Preferably, the two zones provide about 1% overlap between the areas of the two zones, about 2% overlap between the areas of the two zones, about 3% overlap between the areas of the two zones, about 3% overlap between the areas of the two zones, about 4% overlap between the areas of the two zones, about 5% overlap between the areas of the two zones, about 10% overlap between the areas of the two zones, about 20% overlap between the areas of the two zones is provided, about 30% overlap between the areas of the two zones is provided, about 40% overlap between the areas of the two zones is provided, about 50% overlap between the areas of the two zones is provided, about 60% overlap between the areas of the two zones is provided, about 70% overlap between the areas of the two zones is provided, about 80% overlap between the areas of the two zones is provided, about 90% overlap between the areas of the two zones is provided or about 100% overlap between the areas of the two zones is provided.

In yet an embodiment of the present invention, the third zone and the fourth zone can be partially overlapping. Preferably the two zones provide about 1% overlap between the areas of the two zones, about 2% overlap between the areas of the two zones, about 3% overlap between the areas of the two zones, about 3% overlap between the areas of the two zones, about 4% overlap between the areas of the two zones, about 5% overlap between the areas of the two zones, about 10% overlap between the areas of the two zones, about 20% overlap between the areas of the two zones is provided, about 30% overlap between the areas of the two zones is provided, about 40% overlap between the areas of the two zones is provided, about 50% overlap between the areas of the two zones is provided, about 60% overlap between the areas of the two zones is provided, about 70% overlap between the areas of the two zones is provided, about 80% overlap between the areas of the two zones is provided, about 90% overlap between the areas of the two zones is provided or about 100% overlap between the areas of the two zones is provided.

Additionally, the third zone can be modified in order to increase or decrease the time of reaction/incubation between the analyte to be analysed or an analogue thereof and the specific binding conjugate and, thereby, increasing the sensitivity of the test.

As mentioned the sample and the specific binding molecule may be retarded by changing the length of the third zone relative to the length of the first, second and fourth zones.

In a preferred embodiment of the present invention the third zone constitute 1-99% of the device according to the present invention, such as 5-90%, e.g 8-75%, such as 10-60%, e.g. 15-50%, such as 20-45%, e.g. 25-40%. In yet an embodiment of the present invention the third zone constitute at the most 99% of device according to the present invention, such as at the most 75%, e.g. at the most 60%, such as at the most 50%, e.g. at the most 40%, such as at the most 30%, e.g. at the most 25%, such as at the most 30%, e.g. at the most 15%, such as at the most 10%, e.g. at the most 5%, such as at the most 1% of the device according to the present invention.

In a preferred embodiment of the present invention the third zone is included in the device as part of at least one of the first zone, second zone and/or fourth zone. In this case the third zone is omitted and the properties, such as slow down of the migration rate, has been incorporated into at least one of the first zone, second zone and/or fourth zone.

In a preferred embodiment of the present invention, the porous material to be used in the third zone is a cellulose material such as product no. 8-S from Schleicher und Schuell.

The Fourth Zone

Additionally, the device comprises a fourth zone. In the present context, the term “fourth zone” relates to a zone wherein at least part of the zone provides a detectable site preferably located downstream from the first zone, second zone and third zone. In an embodiment of the present invention the fourth zone comprises more than one detectable site. When more than one detectable site is present the detectable sites can be partially overlapping or they can be completely separated. The detectable site or the detectable sites can form part of the fourth zone or can be a separate material connected to the fourth zone.

In the present context the term “detectable site” relates to the incorporation in at least part of the fourth zone an immobilised state the same type of analyte as the one to be assayed or an analogue thereof being capable of specifically binding to the non-immobilised molecule.

In a preferred embodiment of the present invention, the porous material to be used in the fourth zone is a cellulose material such as product no. HF090 from Millipore.

In one preferred embodiment of the present invention, a partial overlapping of more than one detectable site are present. Preferably, the at least two sites provide about 1% overlap between the areas of the sites, about 2% overlap between the areas of the sites, about 3% overlap between the areas of the sites, about 4% overlap between the areas of the sites, about 5% overlap between the areas of the sites, about 10% overlap between the areas of the sites, about 20% overlap between the areas of the sites is provided, about 30% overlap between the areas of the sites is provided, about 40% overlap between the areas of the sites is provided, about 50% overlap between the areas of the sites is provided, about 60% overlap between the areas of the sites is provided, about 70% overlap between the areas of the sites is provided, about 80% overlap between the areas of the sites is provided, about 90% overlap between the areas of the sites is provided or about 100% overlap between the areas of the sites is provided. When two or more detectable sites are overlapping the label selected to to be coupled to the specific binding molecule for one analyte is different from the label selected to be coupled to another specific binding molecule for another analyte, thereby retrieving different signals.

The fourth zone has immobilised thereto in at least part of the zone the same kind of analyte as the one to be assayed or an analogue thereof. In the present context, the term “the same kind of analyte as the one to be assayed” relates to any compound comprising the same features and/or the same structure as the analyte to be assayed, and in the present context “an analogue thereof” relates to any molecule or compound capable of performing the same specific binding as the analyte to be assayed.

In the present context, the term “same features as the analyte to be assayed” relates to any compound capable of providing a specific binding similar to the binding performed by the analyte to be assayed and any compound having the same physically and chemically features as the analyte to be assayed.

In the present context, the term “same structure as the analyte to be assayed” relates to any compound having the same physical and chemical structure as the analyte to be assayed.

In a preferred embodiment of the present invention, the device is provided without any internal reference site. In such cases an external reference may be used for performing the quantification of the analyte to be assayed. In the present context the term “external reference” relates to a reference used as a calibration, value reference, information object, etc. for the analyte to be assayed and which has been excluded from the device of the present invention.

In another preferred embodiment of the present invention, the device is provided with an internal reference site. In such cases the internal reference may be used for performing the quantification of the analyte to be assayed. In the present context the term “internal reference” relates to a reference used as a calibration, value reference, information object, etc. for the analyte to be assayed and which has been incorporated into the device and preferably into the fourth zone.

In an embodiment of the present invention, the second zone incorporating a non-immobilised second molecule capable of specific binding to a compound different from the analyte to be assayed and incapable of specific binding to the analyte to be assayed, said specific binding molecule is provided with a detectable label.

In yet a preferred embodiment of the present invention the fourth zone incorporating in at least part of the zone an immobilised state a compound different from the analyte to be assayed and capable of specific binding the non-immobilised second molecule, said compound is incapable of binding specifically to the non-immobilised molecule capable of specifically binding to the analyte. In this manner the immobilised compound capable of binding the non-immobilised second molecule provides a reference site preferably located in the fourth zone.

The reference site can be designed merely to convey an unrelated signal to the user that the device has worked or the reference site can be used as a calibration in order to increase the sensitivity of the device. When used as a functional indicator the reference site can contain an anhydrous reagent that, when moistened, produces a colour change or colour formation, e.g. anhydrous copper sulphate which will turn blue when moistened by an aqueous sample. As a further alternative, a reference site could contain immobilised analyte which will react with excess specific binding molecule from the second zone. As the purpose of the reference site is to indicate to the user that the test has been completed and/or to count for variations in the device, the reference site should be located downstream from the detection site in which the desired test result is recorded.

In the present context the term “second specific binding molecule” used interchangeable with the terms “non-immobilised second molecule” and “non-immobilised second specific binding molecule” relates to a molecule of a reference pair. In the present context, the term “reference pair” refers to, two molecules where the molecules through chemical or physical means specifically bind to each other and the two molecules are capable of binding each other with substantially no interference with either the analyte in the sample or the non-immobilised specific binding molecule. Preferably, the reference pair is selected from the group consisting of protein-antibody, antigen-antibody, antibody-antibody, lectin-carbohydrate, hormone-antibody and hormone-receptor all providing a specific binding symbolised by a line (−).

In yet an embodiment of the present invention, the antibodies used as reference pair are either monoclonal antibodies or polyclonal antibodies and is produced in a mammal selected from the group comprising mouse, rats, rabbit, hamster, goat, sheep, cow and horse. As an example the reference pair comprise an anti-goat antibody immobilised in the fourth zone and antibodies from goats in the second specific binding molecule in the second zone.

In a preferred embodiment of the present invention, the ratio between non-immobilised second molecule capable of binding to a compound different from the analyte to be assayed and the detectable label is in the range of 1:99 respectively, such as in the range of 25:75 respectively, e.g. in the range of 50:50, such as in the range of 75:25 respectively and e.g. in the range of 99:1 respectively.

In a preferred embodiment of the present invention, at least one of the detection site(s) and/or the reference site forms part of the fourth zone.

In an embodiment of the present invention, at least one of the detection site(s) and/or the reference site comprises individual sites connected to the fourth zone.

The molecule similar to the analyte or analogue thereof and/or the second molecule different from the analyte or analogue to be assayed, immobilised in at least part of the fourth zone may be immobilised by direct attachment to the porous material. Preferably, the immobilisation is performed by using a molecular spacer. In the present context, “molecular spacer” refers to a molecule or a compound capable of linking a molecule similar to the analyte or analogue thereof and/or the second molecule different from the analyte or analogue to be assayed to the fourth zone. In this way, the molecular spacer comprises a molecule or a compound capable of extending the distance between the detection site and reference site and said molecule and compound and makes it more accessible to react with the specific binding molecule and/or second specific binding molecule.

In a preferred embodiment of the present invention, the spacer molecule is selected from the group consisting of proteins, peptides, polypeptides, amino acids and small organic molecules. Preferably the spacer molecule is bovine serum albumin (BSA).

In an embodiment of the present invention the spacer molecule and the analyte or analogue being immobilised to the fourth zone are coupled using carboxymethyloxime (CMO) and/or hemisuccinyliodohistamine (HMS). Both the CMO-coupling and the HMS-coupling is performed by traditional procedures known in the art.

In an embodiment of the present invention the ratio between BSA and the analyte or analogue being immobilised and/or the ration between BSA and the compound to be immobilised is e.g. 1:99, such as 25:75, e.g. 50:50, such as 75:25, e.g. 99:1. Preferably the ratio between BSA and the analyte or analogue being immobilised and/or the ration between BSA and the compound to be immobilised is within the ratio of 10:1, such as 7.5:1, e.g. 5:1, such as 2.5:1, e.g. 0.5:1.

In yet an embodiment of the present invention, the fourth zone is provided with at least one detectable site to provide a device for detecting at least one analyte in a liquid sample. In another embodiment of the present invention the total number of detectable sites in the device of the present invention is at least 2 sites, such as 3 sites, for instance 4 sites, such as 5 sites, for instance 6 sites, such as 10 sites, for instance 15 sites, such as 30 sites, for instance 50 sites. Here, it is possible by simultaneously assaying a multiplicity of analytes to retrieve more analytical data from one sample in one assay run and by the use of a single assaying device.

It is obvious that the device according to the present invention can have all kinds of shapes such as circularly shaped, conically shaped or shaped as a triangle, where at least two fourth zones are located around the centre of the device whose centre may act as an first zone.

The Fifth Zone

Additionally, the device according to the present invention is provided with a fifth zone. In the present context, the term “fifth zone” refers to a zone located downstream from the fourth zone and is selected from the group of porous materials capable of increasing capillary effect in the other zones present in the device by providing force to the sample to direct the migration from the first zone to the second zone and through the third zone, fourth zone to the end in the fifth zone. The fifth zone also provides a sponge effect by adsorbing the sample that has passed through the assay device into which fifth zone sample not detained during migration may be absorbed.

The fifth zone should provide sufficient absorptive capacity to allow any unbound specific binding molecule to wash out from the fourth zone.

In an embodiment of the present invention, the fifth zone and the fourth zone can be partially overlapping. Preferably the two zones provide about 1% overlap between the areas of the two zones, about 2% overlap between the areas of the two zones, about 3% overlap between the areas of the two zones, about 3% overlap between the areas of the two zones, about 4% overlap between the areas of the two zones, about 5% overlap between the areas of the two zones, about 10% overlap between the areas of the two zones, about 20% overlap between the areas of the two zones is provided, about 30% overlap between the areas of the two zones is provided, about 40% overlap between the areas of the two zones is provided, about 50% overlap between the areas of the two zones is provided, about 60% overlap between the areas of the two zones is provided, about 70% overlap between the areas of the two zones is provided, about 80% overlap between the areas of the two zones is provided, about 90% overlap between the areas of the two zones is provided or about 100% overlap between the areas of the two zones is provided.

In a preferred embodiment of the present invention, the porous material to be used in the second zone is a cellulose material such as product no. D28 from Whatman.

Calibration Zone

In a preferred embodiment of the present invention a calibration zone is located downstream from the fourth zone and upstream from the fifth zone.

In a preferred embodiment, the calibration zone comprises an immbilised binding agent having an affinity for the labelled non-immobilised molecule capable of binding to the analyte to be assayed. The binding agent will capture any labelled non-immobilised molecule which has not been captured in the fourth zone upstream from the calibration zone. In operation, the presence of the labelled non-immobilised molecule in the calibration zone indicates that adsorptive transport has operated properly and/or the signals obtained from the fourth zone and the calibration zone are used for the calculation of the amount of analyte present in the sample.

If a calibration zone is present, the unbound complex or the free labelled non-immobilised molecule will accumulate in the calibration zone.

In yet a preferred embodiment, the calibration zone has immobilised thereon polyclonal or monoclonal antisera specific for the labelled non-immobilised molecule. The appearance of colour from the calibration zone indicates proper functioning of the test, irrespective of the presence or absence of analyte in the sample.

The Solid Support

The device according to the present invention, may be supported by a solid support. In the present context, the term “solid support” refers to a material, which has no influence on the migration or on the reaction of the liquid sample or on the specific binding molecule. The solid support provides a stabilising basis for the assay device and provides sufficient strength to maintain the desired physical shape and has substantially no interference with the production of a detectable signal.

In an embodiment of the present invention, the material for the solid support is selected from the group consisting of tubes, polymeric beads, nitrocellulose strips, membranes, filters, plastic shets and the like. Naturally, synthetic and natural occurring materials that are synthetically modified can be used as the material of the solid phase. Such materials include polysaccharides, for instance cellulosic materials such as paper and cellulosic derivatives, such as cellulose acetate and nitrocellulose, silica, inorganic materials, such as, for example, deactivated alumina, diatomaceous earth, MgSO₄ or other inorganic finely divided material uniformly dispersed in a porous polymeric matrix, wherein the matrix may comprise one or more polymers such as homopolymers and copolymers of vinyl chloride, for instance, polyvinyl chloride, vinyl chloride-propylene copolymer, and vinyl chloride-vinyl acetate copolymer, cloth, both naturally occurring (for instance, cotton) and synthetic (for instance, nylon), porous gels, such as silica gel, agarose, dextran, and gelatin, polymeric films, such as polyacrylamide, and the like.

In a preferred embodiment of the present invention, the material to be used in as solid support is a nitrocellulose membrane, such as Millipore HF090, pre-laminated cards, Mylar backed or 3M Transperant Diagnostic Tape 9843R.

Analytes to be determined

A device or a method based on the above principles can be used to determine a wide range of analytes by choice of an appropriate specific binding molecule, and the invention is not limited to examples mentioned herein. The analytes to be assayed can be selected from the group consisting of proteins, haptens, immunoglobulins, antibodies, hormones, polynucleotides, steroids, drugs, and infectious disease agents such as bacteria.

In the present context, the term “steroid” refers to a group of chemical substances related to one another in structure and each containing the same kind of chemical skeleton comprising a tetracyclic cyclopenta[a]phenanthrene or a cyclopentanoperhydroxy-phenanthrene ring system or being a derivative compound of cholesterol.

In an embodiment of the present invention, the group of chemical compounds to be assayed is selected from the group consisting of an adrenocorticoid, a progestagen, an estrogen and an androgen. Non-limiting examples of steroids to be assayed according to the present invention include pregnenolone, progesterone, testosterone, dihydrotestosterone, estrone, estradiol, cortisol, cortisone, aldosterone, corticosterone, androstenedione, 17α-OH-pregnenolone, 17α-OH-progesterone, 11-desoxy-corticosterone, 11-desoxycortisol and dehydroepiandrosterone.

In one embodiment of the present invention, the steroid to be assayed is progesterone. Preferably, progesterone is assayed in a liquid sample containing progesterone in concentrations of at most 100 ng/ml, at most 75 ng/ml, at most 50 ng/ml, for instance at most 30 ng/ml, such as at most 20 ng/ml, for instance at most 15 ng/ml, such as at most 10 ng/ml, for instance at most 5 ng/ml, such as at most 1 ng/ml, for instance at most 0.5 ng/ml, such as at most 0.1 ng/ml. Preferably progesterone is assayed in a liquid sample containing progesterone in concentrations in the range of 0-100 ng-ml, such as 0-50 ng/ml, e.g. 0-40 ng/ml, such as 0-30 ng/ml, e.g. 0-20 ng/ml, such as 0-15 ng/ml, e.g. 010 ng/ml, such as 0-8 ng/ml, e.g. 0-5 ng/ml.

Preferably, progesterone is detected in an assay within the range of 0-5 ng/ml wherein the standard deviation for accuracy, repeatability, reproducibility and/or batch to batch variation is at the most 2 ng/ml, such as at the most 1.5 ng/ml, e.g. at the most 1 ng/ml, such as at the most 0.75 ng/ml, e.g. at the most 0.5 ng/ml, such as at the most 0.25 ng/ml, e.g. at the most 0.1 ng/ml.

Preferably, progesterone is detected in an assay within the range of 5-15 ng/ml wherein the standard deviation for accuracy, repeatability, reproducibility and/or batch to batch variation is at the most 5 ng/ml, such as at the most 4 ng/ml, e.g. at the most 3 ng/ml, such as at the most 2 ng/ml, e.g. at the most 1.5 ng/ml, such as at the most 1 ng/ml, e.g. at the most 0.5 ng/ml, such as at the most 0.1 ng/ml.

Preferably, progesterone is detected in an assay within the range of 15-100 ng/ml wherein the standard deviation for accuracy, repeatability, reproducibility and/or batch to batch variation is at the most 15 ng/ml, such as at the most 10 ng/ml, e.g. at the most 7 ng/ml, such as at the most 6 ng/ml, e.g. at the most 5 ng/ml, such as at the most 4 ng/ml, e.g. at the most 3 ng/ml, such as at the most 2 ng/ml, e.g. at the most 1 ng/ml, such as at the most 0.5 ng/ml, e.g. at the most 0.1 ng/ml

In another embodiment of the present invention, the steroid to be assayed is estradiol.

Preferably, estradiol is assayed in a liquid sample containing estradiol in concentrations of at most 5 ng/ml, for instance at most 3 ng/ml, such as at most 2 ng/ml, for instance at most 1.5 ng/ml, such as at most 1 ng/ml, for instance at most 0.5 ng/ml, such as at most 0.1 ng/ml, for instance at most 0.05 ng/ml, such as at most 0.01 ng/ml.

Accordingly, in a further embodiment of the present invention, the steroid to be assayed is testosterone. Preferably testosterone is assayed in a liquid sample containing testosterone in concentrations of at most 20 ng/ml, for instance at most 10 ng/ml, such as at most 8 ng/ml, for instance at most 6 ng/ml, such as at most 5 ng/ml, for instance at most 4 ng/ml, such as at most 3 ng/ml, for instance at most 1 ng/ml, such as at most 0.5 ng/ml.

Samples to be Analysed

In the present context the term “a sample” relates to any sample found in the form of liquid, solid or gas and which is liquefied at the time of assaying. In order to wet the porous material used in the first zone, second zone, third zone, fourth zone and fifth zone which permits migration, a liquid sample is applied. Additionally a minimum of handling steps, of the liquid sample is necessary before applying it to the first zone. In the present context, the term “handling steps” relates to any kind of pre-treatment of the liquid sample before or after it has been applied to the assay device. This pre-treatment comprises separation, filtration, dilution, distillation, concentration, inactivation of interfering compounds, centrifugation, heating, fixation, addition of reagents, or chemical treatment.

In a preferred embodiment of the present invention, the sample is collected from any kind of mammal, preferably a mammal selected from the group consisting of herd animals, cows, camels, buffaloes, pigs, horses, deer, sheep, goats, pets, dogs, cats and humans.

In a preferred embodiment of the present invention, the sample can be derived from any desirable source such as physiological fluids. Preferably, this source is selected from the group consisting of milk, blood, serum, plasma, saliva, urine, sweat, ocular lens fluid, cerebral spinal fluid, ascites fluid, mucous fluid, synovial fluid, peritoneal fluid, amniotic fluid or the like.

Besides physiological fluids, other liquid samples such as various water samples, food products and the like can be used. In addition, a solid test sample can be used once it is modified to form a liquid sample, for instance in the form of a solution, a suspension or an emulsion.

The Ancillary Compound

Because of the complexity of the liquid samples to be assayed in the present invention it may occasionally be an advantages to use an ancillary compound in order to improve the flow of the liquid sample through the zones and to provide a fast, consistent and quantitative release of the non-immobilised specific binding molecule. The ancillary compound may be supplied to the device either by a) adding it to the first zone alone or together with the liquid sample, b) incorporating the ancillary compound into at least one of the first zone, second zone, third zone, fourth zone and/or fifth zone, or c) a combination thereof.

In an embodiment of the present invention, the ancillary compound is added to the first zone before the liquid sample is added and the ancillary compound is maintained in a moistened state.

In another preferred embodiment of the present invention, the ancillary compound and the liquid sample are added to the first zone in layers. In the present context, the term “layers” refers to the splitting up of the volume of the ancillary compound and the volume of the liquid sample, and then the ancillary compound and the liquid sample is added to the first zone one after another. In this case, the ancillary compound may be added as a liquid as well as a solid compound. In an embodiment of the present invention, the ancillary compound and the liquid sample is split into at least 2 volumes each providing 4 alternating layers of ancillary compound and liquid sample, e.g. the ancillary compound and the liquid sample is split into at least 3 volumes each providing 6 alternating layers of ancillary compound and liquid sample, such as the ancillary compound and the liquid sample is split into at least 4 volumes each providing 8 alternating layers of ancillary compound and liquid sample, e.g. the ancillary compound and the liquid sample is split into at least 6 volumes each providing 12 alternating layers of ancillary compound and liquid sample, such as the ancillary compound and the liquid sample is split into at least 8 volumes each providing 16 alternating layers of ancillary compound and liquid sample, e.g. the ancillary compound and the liquid sample is split into at least 10 volumes each providing 20 alternating layers of ancillary compound and liquid sample, such as the ancillary compound and the liquid sample is split into at least 20 volumes each providing 40 alternating layers of ancillary compound and liquid sample.

In yet an embodiment of the present invention, the ancillary compound decreases non-specific binding of the analyte and non specific binding of the non-immobilised specific binding molecule.

Additionally, the ancillary compound provides low affinity for unspecific protein binding.

In another embodiment of the present invention, the ancillary compound provides low retention of triglyceride rich samples.

Additionally, the ancillary compound decreases the viscosity of the liquid sample.

In an embodiment of the present invention the ancillary compound contains chemical constituents selected from the group consisting of water, surfactant, salt, acid, base, metals, sugar, proteins and lipid.

In a further embodiment, improved flow of the liquid sample and fast, consistent and quantitative release of the non-immobilised specific binding conjugate can be accomplished by applying an ancillary compound to at least one of the first zone, the second zone, the third zone, the fourth zone or fifth zone comprising at least one ancillary compound capable of improving the flow of the liquid sample.

Useful Embodiments

In an embodiment the present invention provides an appliance comprising a multiplicity of devices applicable for an automatic, a semi-automatic and a continuous system. This kind of system could be, but is not limited to, the system disclosed in the co-pending European patent application No. 01610022.4. In the present context, the term “appliance” relates to an apparatus which constantly provides devices according to the invention for the detection of the analyte to be assayed and this provision proceeds without manual operations. The appliance according to the present invention is selected from the group consisting of a strip, a band, a tape and a film.

The device according to the present invention may be applied for different purposes and used for measuring in various types of samples including physiological fluids, organic fluids and naturally occurring fluids for detection and quantification of analytes.

A device according to the present invention may be used for measuring different physiological fluids. The device may be used as a pregnancy test where the liquid sample used can be either blood or urine. Additionally the device may be used for detecting diseases and infections, for instance caused by microorganisms such as bacteria and yeast, or other states of abnormal physiological states in a mammal. These physiological states can include the detection of analytes that are combined with compounds related to the consumption of illegal substances for improvement of for instance physical performance. Furthermore, the device may be used on the street for measuring the content of alcohol without taking blood samples, but by using for instance saliva.

The device of the present invention may be used in medical diagnostics, for instance in a laboratory, at the hospital, at the doctor or in an ambulance. The device can also be applied to measure different analytes in drinking water, wastewater, seawater and ground water.

It is advantageous that the device of the present invention can be used without the use of additional reagents, and the device can be used by a person not skilled in the art or by a person not having the professional skills usually required in order to perform the analysis.

The device according to the present invention may also be applied in the analysis of food products, to control the content of for instance microorganisms, growth hormones and antibiotics.

The device can be compatible with automated or semi-automatic systems such as optimising performances of milk producing herds when the assayed analyte is a steroid.

It is obvious that the device and/or the method according to the present invention may be combined with one or more of other pregnancy prediction devices and/or methods which is already known by the person skilled in the art such as an activity measurement.

The following non-limiting drawings, embodiments and examples will illustrate the invention further.

FIG. 1 shows the level of progesterone during the cow reproduction cycle. The solid line symbolises the level of progesterone in successfully inseminated cows, and the dashed line symbolises the unsuccessfully inseminated cows.

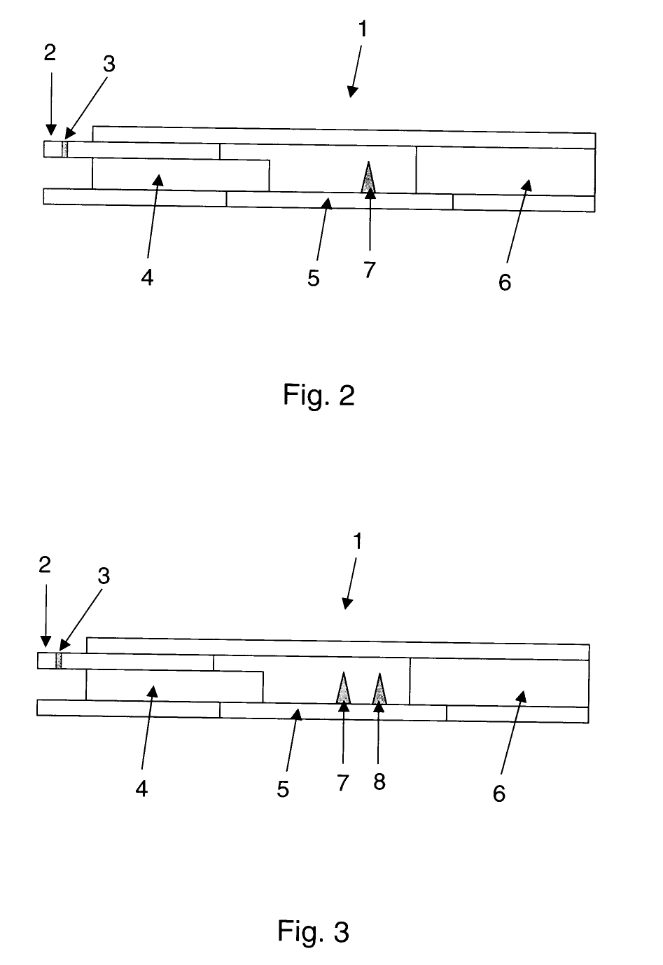

FIG. 2 shows an exemplary embodiment of the assay device according to the invention without an internal reference.

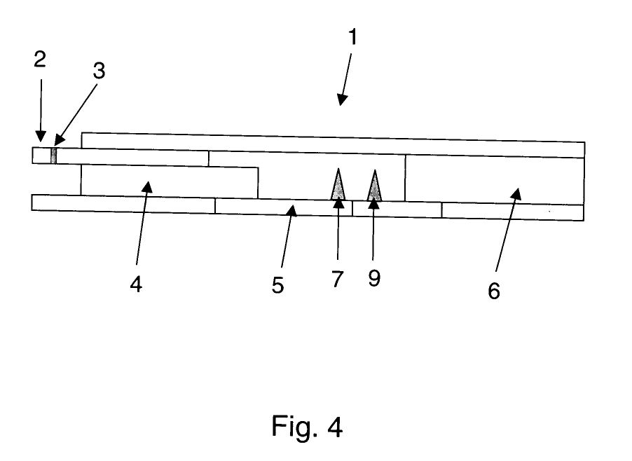

FIG. 3 shows an exemplary embodiment of the assay device with an internal reference according to the invention.

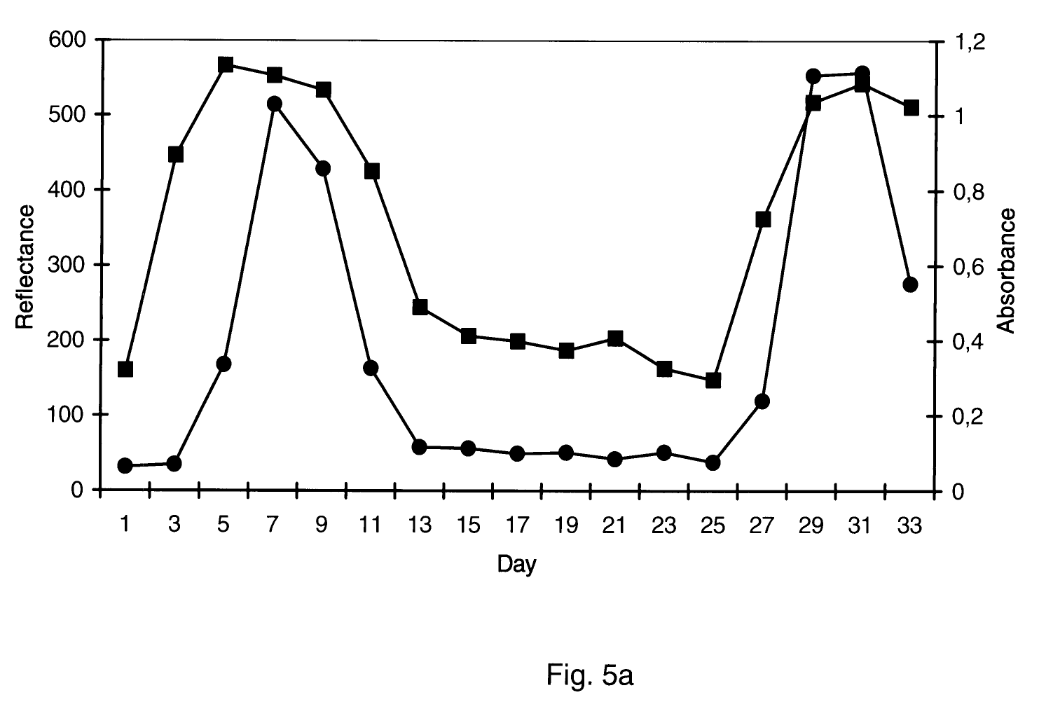

FIG. 4 shows an exemplary embodiment of the assay device with a calibration zone according to the invention.

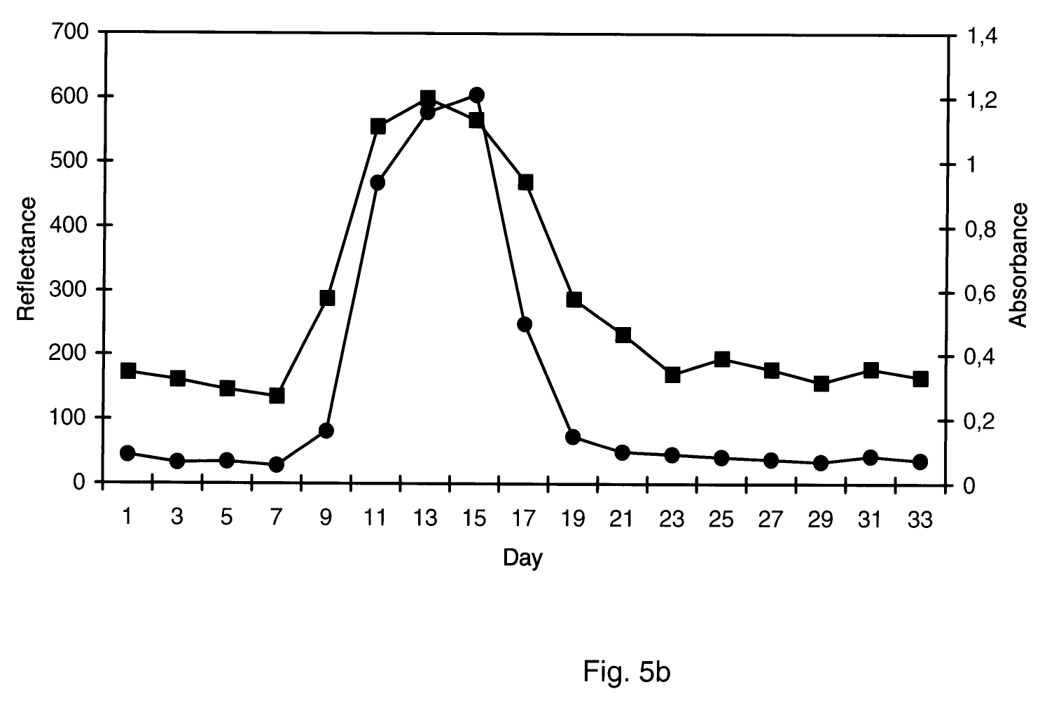

FIG. 5a shows measurement during an oestrus cycle of raw milk samples collected from a cow after an unsuccessful insemination and the cow enters a new hormone cycle. Shown is raw data from the experiments performed with the wet chemical ELISA reference assay (squares) and the developed progesterone device of the present invention (circles), respectively. Thus, high values of absorbance/reflectance reflect low progesterone concentrations and vice versa.

FIG. 5b shows measurement during an oestrus cycle of raw milk samples collected from a cow after a successful insemination and the cow becomes pregnant. Shown is raw data from the experiments performed with the wet chemical ELISA reference assay (squares) and the developed progesterone device of the present invention (circles), respectively. Thus, high values of absorbance/reflectance reflect low progesterone concentrations and vice versa.

### DESCRIPTION OF THE PREFERRED EMBODIMENTS

The first preferred embodiment

FIG. 2 represents a schematic diagram of the immunoassay provided without an internal reference within device according to the invention.

Referring to FIG. 2, the device (1) having in one end a first zone (2) connected to a second zone (3) in which a specific binding molecule reactive to the analyte to be measured is located and for providing detectable signals the specific binding molecule is coupled to a label. The second zone (3) is connected via a third zone (4) and a fourth zone (5) to a fifth zone (6) in the other end of the device (1). The fourth zone (5) comprises a detection site (7) located downstream of the third zone (4). In the said detection site (7), an analyte of the same type as the analyte to be assayed or an analogue thereof is immobilised.

In operation, the liquid sample is added at the first zone (2), the liquid sample makes the first zone (2) and the second zone (3) wet and thereby releases the specific binding molecule found in the second zone (3). The specific binding molecule consists of an analyte specific antibody coupled to a detectable label, for instance small gold particles. While the liquid sample and the specific binding molecule migrate through the third zone (4) and the fourth zone (5), immobilised analyte in the detection site (7) of the same type as the analyte to be assayed or an analogue thereof will bind the specific binding molecule which have not already been bound by the analyte in the liquid sample and the amount of detectable label bound to the detection site (6) will be inversely proportional to the concentration of analyte in the liquid sample and may be determined by the use of an external reference.

It will be seen from the above description that there are many variations that can be made in respect of reagents, materials, analytes, rearrangements of zones and the like, all without leaving the scope of the present invention.

The Second Preferred Embodiment

FIG. 3 represents a schematic diagram of the immunoassay provided with an internal reference within device according to the invention.

Referring to FIG. 3, the device (1) comprises a first zone (2) connected to a second zone (3) in which a non-immobilised specific binding molecule reactive to the analyte to be measured and a non-immobilised second molecule capable of binding specifically to a compound different from the analyte to be assayed are located. The second zone (3) is connected via a third zone (4) and a fourth zone (5) to a fifth zone (6) in the other end of the device (1). The said fourth zone (5) comprises a detectable site (7) and a reference site (8) and the reference site (8) is located downstream of the detectable site (7). In the said detectable site (7) an analyte of the same type as the analyte to be assayed is immobilised. In the said reference site (8) a compound different from the analyte to be assayed and which is capable of binding specifically to the non-immobilised second molecule is immobilised. For providing detectable signals the specific binding molecule is coupled to a label and this label might be the same or be different from the label coupled to the non-immobilised second specific binding molecule.

In operation, the liquid sample is added at the first zone (2), the liquid sample makes the first zone (2) and the second zone (3) wet and thereby releases the non-immobilised specific binding molecule and the non-immobilised second specific binding molecule, found in the second zone (3). The specific binding molecule is coupled to a detectable label for instance small gold particle, the second specific binding molecule consists of a compound different from the analyte to be assayed and a detectable label for instance also a small gold particle. While the liquid sample and the specific binding molecule and the second specific binding molecule, migrate through the fourth zone (5), immobilised analyte in the detection site (7) of the same type as the analyte to be assayed will bind the specific binding molecule which have not already been bound by the analyte in the liquid sample and the amount of detectable label bound to the detectable site (7) will be inversely proportional to the concentration of analyte in the liquid sample.

Given the fact that the quality of the porous material used in the different zones may be quite variable, resulting in a variable capillary flow rate (e.g. ±33%) and thereby a variable detectable signal at the detectable site (7), an internal reference system in the form of a reference site (8) is included in the test strips. The internal reference system consists of a second specific molecules such as antibodies conjugated to for instance gold particles which are also provided in the second zone (3) and a compound specific for said second specific binding molecule is immobilised in at least part of the fourth zone (5) and the result will be made by taking a ratio or by using a specially designed algorithm of the signals at the detection site (7) and at the reference site (8).

The Third Preferred Embodiment

FIG. 4 represents a schematic diagram of the immunoassay provided with a calibration zone within device according to the invention.

Referring to FIG. 4, the device (1) comprises a first zone (2) connected to a second zone (3) in which a non-immobilised specific binding molecule reactive to the analyte to be measured. The second zone (3) is connected via a third zone (4) and a fourth zone (5) to the calibration zone (9) which finally is connected to a fifth zone (6) in the other end of the device (1). The said fourth zone (5) comprises a detectable site (7) wherein an analyte of the same type as the analyte to be assayed is immobilised. In the calibration zone (9) an agent having an affinity for the labelled non-immobilised molecule capable of binding to the analyte to be assayed is immobilised.

In operation, the liquid sample is added at the first zone (2), the liquid sample makes the first zone (2) and the second zone (3) wet and thereby releases the non-immobilised specific binding molecule found in the second zone (3). The specific binding molecule is coupled to a detectable label for instance small gold particle. While the liquid sample and the specific binding molecule migrate through the third zone (4) and fourth zone (5), immobilised analyte in the detection site (7) of the same type as the analyte to be assayed will bind the specific binding molecule which have not already been bound by the analyte in the liquid sample. The labelled non-immobilised molecule which has not been captured in the fourth zone (5) upstream from the calibration zone (9) and the labelled non-immobilised molecule which has been bound to the analyte in the sample will be captured by the binding agent immobilised in the calibration zone (9).

Using the calibration zone (9) is another way of take in consideration the influence of the material used in the device and the influence of the device as such.

It will be seen from the above description that there are many variations that can be made in respect of reagents, materials, analytes, rearrangements of zones and the like, all without leaving the scope of the present invention.

### EXAMPLES

### Example 1

The purpose of this experiment is to illustrate that the of the device of the present invention was able to measure small variations in progesterone concentrations quantitatively in milk samples collected during an oestrus cycle and that it is possible to 20 measure small variations in the progesterone concentrations. Thus, to show it is possible with the developed device to identify heat and pregnancy/non-pregnancy

Method

20 μl of milk and 100 μl of ancillary compound is added simultaneously to the first zone of the progesterone dry sticks. The sticks are incubated 5 min at room temperature and subsequently read in a designated reader measuring reflectance at 420 nm.

Results

Obtained results are shown in FIGS. 5a and 5b where the adsorbance and reflectance measured using the ELISA reference assay and the developed progesterone assay is plotted against day of collecting the milk sample. As can be seen, the developed device is able to make quantitative measurements of progesterone similar of what could be obtained using the ELISA assay. Also, it was possible with the developed device to identify heat and to differentiate of whether the insemination of the cow is successful and the cow becomes pregnant (FIG. 5b) or unsuccessful and the cow enters a new hormone cycle (FIG. 5a).

### REFERENCES

Abeyawardene, S. A., Hathorn, D. J., Glencross, R. G., 1984. Concentrations of oestradiol-17b and progesterone in bovine plasma and defatted milk during the post-partum anovulatory period, during oestrous cycles and following ovariectomy. Br. vet. J. 140, 458-467.

Royal, M. D., Darwash, A. O., Flint, A. P. F., Webb, R., Woolliams, J. A., Lamming, G. E., 2000. Declining fertility in dairy cattle: changes in traditional and endocrine parameters of fertility. Animal Science 70, 487-501.

Salisbury, G. W., VanDemark, N. L., 1961. Physiology of reproduction and artificial insemination of cattle. 1-639. 1961. W. H. Freeman and Co.

Waldmann, A., 1993. Enzyme immunoassay (EIA) for milk progesterone using a monoclonal antibody. Animal Reproduction Science 34, 19-30.

EP 0 810 436

U.S. Pat. No. 6,001,658

EP 0 895 084

EP 1 061 369

1. An analytical device comprising a porous material that permits liquid to migrate therein, the device comprising in the migration direction:
(i) a first zone onto which a sample suspected of containing an analyte to be assayed can be applied,
(ii) a second zone incorporating a non-immobilised molecule capable of specifically binding to the analyte, said non-immobilised molecule is provided with a detectable label,
(iii) a third zone capable of retarding the rate of migration of the sample and the non-immobilised molecule, and
(iv) a fourth zone incorporating in at least part of the zone an immobilised state the same type of analyte as the one to be assayed or an analogue thereof being capable of specifically binding to the non-immobilised molecule.

2. A device according to claim 1 wherein the first zone and the second zone are overlapping.

3. A device according to any of the preceding claims wherein the second zone and the third zone are overlapping.

4. A device according to any of the preceding claims wherein the third zone and the fourth zone are overlapping.

5. A device according to any of the preceding claims wherein the device comprises a fifth zone into which sample not detained during migration may be adsorbed.

6. A device according to claim 5 wherein the fourth zone and the fifth zone are overlapping.

7. A device according to any of the preceding claims wherein the second zone incorporating a non-immobilised second molecule capable of binding to a compound different from the analyte to be assayed and incapable of specific binding to the analyte to be assayed, said molecule is provided with a detectable label.

8. A device according to claim 7 wherein the fourth zone incorporating in at least part of the zone an immobilised state a compound different from the analyte to be assayed and capable of binding the non-immobilised second molecule, said compound is incapable of binding specifically to the non-immobilised molecule capable of specifically binding to the analyte.

9. A device according to any of the preceding claims wherein the molecule in the second zone is selected from the group consisting of antibodies and receptors.

10. A method according to any of the preceding claims wherein the first zone, the second zone, the third zone, the fourth zone and the fifth zone comprises of a porous material, said porous material is selected from the group consisting of a nitrocellulose membrane, cellulose, a polymer such as nylon, polyvinylidene fluoride or latex, glass fibre, woven fibres, non-woven fibres and a chromatographic gel membrane.

11. A method according to claim 10 wherein the average pore size of the porous material is in the range of 10-10.000 nm.

12. A method according to any of claims 10-11 wherein the capacity of the porous material to bind proteins is in the range of 1-400 μg/cm².

13. A method according to any of claims 10-12 wherein the capillary flow-rate of the porous material is in the range of 50-250 sec/4 cm.

14. A device according to any of the preceding claims wherein the analyte or analogue being immobilised to the fourth zone through a space molecule

15. A device according to claim 14 wherein the spacer molecule is selected from the group consisting of a peptide, a polypeptide and a protein.

16. A device according to any of the claims 14 or 15 wherein the spacer molecule is bovine serum albumin.

17. A device according to any of the claims 15 or 16 wherein the spacer molecule and the analyte or analogue being immobilised to the fourth zone is coupled using CMO and/or HMS.

18. A device according to any of the proceding claims wherein the capability of retarding the sample and the specific binding molecule of the third zone is provided by changing the length of the porous material used in the third zone, changing the porosity of the porous material and/or adding at least one substance.

19. A device according to claim 18 wherein the sample and the specific binding molecule is retarded by changing the length of the third zone relative to the length of the first, second and fourth zones.

20. A device according to any of the preceding claims wherein the third zone constitute 199% of the porous material used in the first zone, second zone, third zone and fourth zone.

21. A device according to any of the preceding claims wherein the device further comprises a calibration zone.

22. A device according to claim 21 wherein the calibration zone is located downstream from the fourth zone and upstream from the fifth zone.

23. A device according to claims 20 or 21 wherein the calibration zone has immobilised thereon polyclonal or monoclonal antisera specific for the labelled non-immobilised molecule capable of binding the analyte to be assayed.

24. A device according to any of the preceding claims wherein at least one zone incorporating at least one ancillary compound capable of improving the flow of the liquid sample.

25. A device according to claim 24 wherein the at ancillary compound is a liquid.

26. A device according to any of claims 24 or 25 wherein the ancillary compound decreases non specific binding of the analyte and non specific binding of the non-immobilised specific binding molecule.

27. A device according to any of the claims 24-26 wherein the ancillary compound provides a fast, consistent and quantitative release of the non-immobilised specific binding molecule.

28. A device according to any of the claims 24-26 wherein the ancillary compound provides low affinity for protein binding.

29. A device according to any of the claims 24-28 wherein the ancillary compound provides low retention of triglyceride rich samples.

30. A device according to any of the claims 24-29 wherein the ancillary compound decreases the viscosity of the sample.

31. A device according to any of the claims 27-30 wherein the ancillary compound contains chemical constituents selected from the group consisting of water, surfactant, salt, acid, base, metals, sugar, proteins and lipid.

32. A device according to any of the preceding claims wherein the device comprises a solid support.

33. A device according to any of the preceding claims wherein said devise is provided in the form of a dry stick.

34. An appliance carrying a multiplicity of the device according to any of claims 1-33.

35. An appliance according to claim 34 wherein an automatic, a semi-automatic and a continuous system is provided.

36. An appliance according to any of claims 34 or 35 wherein the appliance is a strip.

37. A method for assaying an analyte in a sample comprising the steps of:
(i) applying the sample suspected of containing an analyte to a first zone,
(ii) permitting the sample to migrate through a second zone incorporating a non-immobilised molecule capable of specifically binding to the analyte, said non-immobilised molecule is provided with a detectable label,
(iii) permitting the sample to migrate through a third zone capable of retarding the rate of migration of the sample and the non-immobilised molecule, and
(iv) permitting the sample to migrate through a fourth zone incorporating in at least part of the zone an immobilised state the same type of analyte as the one to be assayed or an analogue thereof being capable of specifically binding to the non-immobilised molecule.

38. A method according to claim 37 wherein at least one ancillary compound capable of improving the flow of the sample is added.

39. A method according to claim 37 wherein at least one ancillary compound is incorporated into at least one zone.

40. A device according to claims 38-39 wherein the ancillary compound decreases non-specific binding of the analyte and non-specific binding of the non-immobilised specific binding molecule.

41. A device according to any of claims 38-40 wherein the ancillary compound provides a fast, consistent and quantitative release of the non-immobilised specific binding molecule.

42. A device according to any of claims 38-41 wherein the ancillary compound provides low affinity for protein binding.

43. A device according to any of claims 38-42 wherein the ancillary compound provides low retention of triglyceride rich samples.

44. A device according to any of claims 38-43 wherein the ancillary compound decreases the viscosity of the sample.

45. A device according to any of claims 38-44 wherein the ancillary compound contains chemical constituents selected from the group consisting of water, surfactant, salt, acid, base, metals, sugar, proteins and lipid.

46. A method according to any of claims 37-45 wherein the analyte to be assayed is a steroid selected from the group consisting of a progestagen, an estrogen and an androgen.

47. A method according to claim 46 wherein the progestagen to be assayed is progesterone.

48. A method according to claim 47 wherein the sample to be assayed is containing 0-50 ng/ml of progesterone.

49. A method according to any of claims 37-48 wherein the specific binding molecule is selected from the group consisting of antibodies and receptors.

50. A method according to claim 49 wherein the antibodies are monoclonal antibodies.

51. A method according to any of claims 37-50 wherein the first zone, the second zone, the third zone, the fourth zone and the fifth zone comprises of a porous material, said porous material is selected from the group consisting of a nitrocellulose membrane, cellulose, a polymer such as nylon, polyvinylidene fluoride or latex, glass fibre, woven fibres, non-woven fibres and a chromatographic gel membrane.

52. A method according to claim 51 wherein the average pore size of the porous material is in the range of 10-10.000 nm.

53. A method according to any of claims 51-52 wherein the capacity of the porous material to bind proteins is in the range of 1-400 μg/cm².

54. A method according to any of claims 51-53 wherein the capillary flow-rate of the porous material is in the range of 50-250 sec/4 cm.

55. A method according to any of claims 37-54 wherein the detectable label is selected from the group consisting of dyes, enzymes, fluorescent compounds, chemiluminescent compounds, radioactive labels and metals.

56. A method according to claim 55 wherein the detectable label is selected from the group consisting of gold, silver, carbon, fluorescent latex beads and dyed latex beads.

57. A method according to any of claims 37-56 wherein the assay time is less than 15 min.

58. A method according to any of claims 37-57 wherein the sample to be assayed is mammalian physiological fluid.

59. A method according to claim 58, wherein the mammalian physiological fluid to be tested is selected from a group consisting of milk samples, urinary samples, blood samples and saliva samples.

60. A method according to any of claims 58-59 wherein the mammal is a cow or a human.

61. A method according to any of claims 37-60, wherein, a device as described in claims 132 and an appliance as described in claims 33-35 is used.

62. A method according to any of the claims 37-61 wherein the capability of retarding the sample and the specific binding molecule of the third zone is provided by changing the length of the porous material used in the third zone, changing the porosity of the porous material and/or adding at least one substance.

63. A device according to claim 62 wherein the sample and the specific binding molecule is retarded by changing the length of the third zone relative to the length of the first, second and fourth zones.

64. A device according to any of the claims 37-63 wherein the third zone constitute 1-99% of the porous material used in the first zone, second zone, third zone and fourth zone.

65. A device according to any of the claims 37-64 wherein the device further comprises a calibration zone.

66. A device according to claim 65 wherein the calibration zone is located downstream from the fourth zone and upstream from the fifth zone.

67. A device according to claims 65 or 66 wherein the calibration zone has immobilised thereon polyclonal or monoclonal antisera specific for the labelled non-immobilised molecule capable of binding the analyte to be assayed.
